(19) United States
(12) Patent Application Publication
(10) Pub. No.: US 2013/0158366 A1
(43) Pub. Date: Jun. 20, 2013

(54) HANDHELD DEVICE FOR FETAL HEALTH MONITORING AND METHOD THEREOF

(71) Applicant: General Electric Company, Schenectady, NY (US)

(72) Inventors: Kiran Bogineni, Bangalore (IN); Fatma Nazan Demirbilek, Milwaukee, WI (US); Nasir Ahmed Desai, Bangalore (IN); Antony Kalugumalai Neethimanickam, Bangalore (IN)

(73) Assignee: GENERAL ELECTRIC COMPANY, Schenectady, NY (US)

(21) Appl. No.: 13/721,543

(22) Filed: Dec. 20, 2012

(30) Foreign Application Priority Data
Dec. 20, 2011 (IN) 4480/CHE/2011

(51) Int. Cl. A61B 5/00; A61B 8/02; A61B 5/01; A61B 5/11

(52) U.S. Cl. CPC A61B 5/4362; A61B 5/11; A61B 8/02; A61B 5/01; A61B 5/746; A61B 5/0002; A61B 5/7475; A61B 5/749; A61B 5/742; A61B 5/0004; A61B 5/74; A61B 5/7246
USPC 600/301

(57) ABSTRACT

A handheld device for fetal health monitoring is provided. The device comprises at least one sensor configured to detect fetal movement, and an ultrasound transducer unit configured to record a heartbeat sound of the fetus. The device further comprises a storage unit connectable to the at least one sensor and the ultrasound transducer unit and configured to store the detected fetal movement along with a timestamp and the heartbeat sound of the fetus in the form of digital information.

### BACKGROUND OF THE INVENTION

The subject matter disclosed herein relates to health monitoring devices. More particularly, the subject matter relates to a handheld device for monitoring health of a fetus.

Perceived fetal movement by a pregnant woman is regarded as an excellent indication of fetal well-being. Fetal movement serves as an indirect measure of the central nervous system's integrity and function. Although fetal movement can be documented by ultrasound as early as 7-8 weeks of pregnancy, the first fetal movement or “flutter” is not usually felt by the pregnant woman until the 16th week (for women who previously delivered a baby) to the 20^{th} week (for women who are pregnant for the first time). Fetal movements generally increase in frequency through pregnancy, particularly at night and when the woman is at rest. At the end of pregnancy (36 weeks and beyond), there is normally a slow change in movements, with fewer violent kicks and more rolling and stretching fetal movements.

Maternal counting and tracking of fetal movements is called kick count. It has been accepted as a cheap, simple, valuable, effective, reliable and harmless screening of fetal well-being in both low and high risk pregnancies. For example, a sudden decrease in fetal movements may indicate potential problems and may need further evaluation of fetal well-being.

Conventionally, fetal kick counts are measured manually by pregnant women. The pregnant woman normally maintains a fetal movement chart where she enters the fetal kick counts along with the timestamp. The conventional method is a burden to pregnant women and it is a time consuming and inaccurate process. If a pregnant woman misses one count then she has to start counting from the beginning.

It is known that maintaining body temperature of pregnant women within optimal limits is very important during pregnancy. If the body temperature of a pregnant woman exceeds the prescribed temperature, then it will affect the fetal well being, hence it is recommended that a pregnant women maintain her body temperature within the optimal limit. The recommended body temperature for the pregnant woman is 102.2° F. Thus, a pregnant woman should not let her core body temperature rise above 102.2° F. However, the preferred temperature may vary from region to region and person to person.

Most women are more tired than usual during the first trimester of pregnancy and toward the end of the third trimester. During early pregnancy, the body's hormones make pregnant women feel sluggish and sleepy. Pregnant women's bodies also realize changes in the way it processes food and nutrients, and it needs to work harder to supply the needs of both the pregnant woman and the developing baby. During the last months of pregnancy, pregnant women have difficulty sleeping and the additional weight of the baby further depletes a pregnant woman's strength and energy. During this time, a pregnant woman requires educational tips for maintaining her mood. Conventionally, pregnant women read bulky books for tips to maintain her mood, know the development of the fetus, and obtain eating tips. The conventional method is a burden to pregnant women and it consumes more time to select correct tips.

During pregnancy pregnant women require assistance by a care giver, a partner, or mid-wife during emergencies. If timely assistance is not provided to a pregnant woman during an emergency, it will affect the fetus, and may result in a still birth. In order to avoid the above problem, pregnant women should be assisted during emergency.

In addition, pregnant women often like to hear their baby's heartbeat and hope to have a good, healthy baby. Normally, the mother would be able to hear the heartbeat of baby only during her monthly checkups.

In light of the forgoing discussion, it is necessary to develop a handheld device which has integrated features such as a fetal movement counter, an ultrasound transducer, a temperature sensor, an alert system and an audio recorder which acts as a personal device for pregnant women for fetal health monitoring.

### BRIEF DESCRIPTION OF THE INVENTION

The shortcomings of the prior art are overcome and additional advantages are provided through the provision of a device and a method as claimed in the present disclosure.

Additional features and advantages are realized through the techniques of the present disclosure. Other embodiments and aspects of the disclosure are described in detail herein and are considered as part of the claimed disclosure.

According to an embodiment of the present invention, a handheld device for fetal health monitoring is provided. The handheld device comprises at least one sensor configured to detect fetal movement, and an ultrasound transducer unit configured to record a heartbeat sound of the fetus. The handheld device further comprises a storage unit connectable to the at least one sensor and configured to store the detected fetal movement along with a timestamp and the heartbeat sound of the fetus in the form of digital information.

According to another embodiment of the present invention, a method of monitoring fetal health is disclosed. The method comprises detecting fetal movement using at least one sensor and recording a heartbeat sound of the fetus using an ultrasound transducer unit. The method further comprises storing the detected fetal movement along with a timestamp and the heartbeat sound of the fetus in the form of digital information in a storage unit.

The foregoing summary is illustrative only and is not intended to be in any way limiting. In addition to the illustrative aspects and features described above, further aspects, and features will become apparent by reference to the drawings and the following detailed description.

### BRIEF DESCRIPTION OF THE DRAWINGS

The novel features and characteristic of the disclosure are set forth in the appended claims. The disclosure itself, however, as well as a preferred mode of use, further objectives and advantages thereof, will best be understood by reference to the following detailed description of an illustrative aspect when read in conjunction with the accompanying figures. One or more aspects are now described, by way of example only, with reference to the accompanying figures wherein like reference numerals represent like elements and in which:

FIG. 1 illustrates an exemplary functional block diagram of a handheld device for fetal health monitoring;

FIG. 2 illustrates a flow chart showing an operational sequence of a fetal motion counting unit of a handheld device according to an embodiment of the present invention;

FIG. 3 illustrates flow chart showing an operational sequence of an ultrasound transducer unit of a handheld device according to an embodiment of the present invention;

FIG. 4 illustrates a display interface with a knowledge database in a handheld device according to an embodiment of the present invention;

FIG. 5 illustrates an alert system of a handheld device according to an embodiment of the present invention;

FIG. 6 illustrates a flow chart showing an operational sequence of an audio recorder of a handheld device according to an embodiment of the present invention;

FIG. 7 illustrates a flow chart showing an operational sequence of a temperature sensor of a handheld device according to an embodiment of the present invention; and

FIG. 8 illustrates a flow chart showing an operational sequence of an alert system of a handheld device according to an embodiment of the present invention.

The figures depict aspects of the disclosure for purposes of illustration only. One skilled in the art will readily recognize from the following description that alternative aspects of the structures and methods illustrated herein may be employed without departing from the principles of the disclosure described herein.

### DETAILED DESCRIPTION OF EXEMPLARY EMBODIMENTS OF THE INVENTION

The foregoing discussion has broadly outlined the features and technical advantages of the present disclosure so that the detailed description of the disclosure that follows may be better understood. Additional features and advantages of the disclosure will be described hereinafter which form the subject of the claims of the disclosure. It should be appreciated by those skilled in the art that the conception and specific aspect disclosed may be readily utilized as a basis for modifying or designing other structures for carrying out the same purposes of the present disclosure. It should also be realized by those skilled in the art that such equivalent constructions do not depart from the spirit and scope of the disclosure as set forth in the appended claims. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

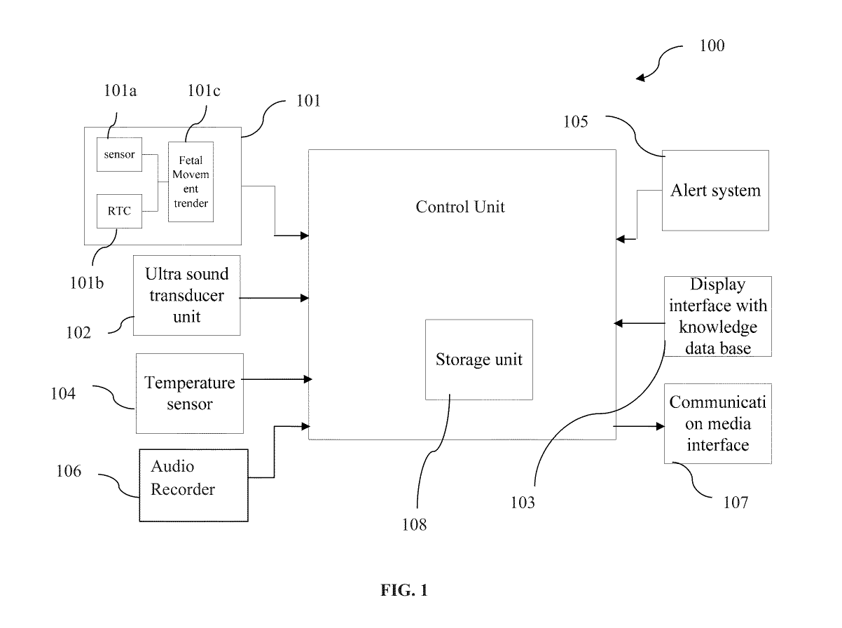

FIG. 1 is an embodiment of the present invention that illustrates a functional block diagram of a handheld device 100 for fetal health monitoring. The handheld device 100 comprises a plurality of components including, but not limited to, a fetal motion count unit 101 for charting fetal movement, an ultrasound transducer unit 102 for recording the heartbeat sound of the fetus and a storage unit 108 for storing the detected fetal motion with timestamp and the heartbeat sound in the form of digital information. The handled device 100 further comprises a temperature sensor 104, for measuring the temperature of the pregnant woman, and an audio recorder 105 and user interface for recording the health conditions of the user. In addition, the handheld device 100 includes an alert system 106 for sending a preconfigured emergency message to a hospital management system or mobile device of a care giver during emergency. A display interface 103 is integrated in the handheld device 100 for displaying the information stored in the knowledge database to educate the user about health tips and to help the user control mood swings. The handheld device 100 is further equipped with a communication media interface for downloading data stored in the storage unit 108.

The fetal motion counting unit 101 provided in the handheld device 100 is employed for counting the movements of the fetus. Fetal movement serves as an indirect measure of the central nervous system integrity and function of the fetus. Generally, it is noticed that a healthy baby moves about 10-15 times in two hours, hence it is very important to measure fetal motions. The fetal motion counting unit 101 comprises a plurality of motion sensors 101a for detecting the movement of the fetus in the womb. The fetal motion counting unit further comprises a fetal movement count trender 101b interfaced with sensors 101a and a real time clock 101c for charting the movement of the fetus along with the timestamp. The fetal movement count trender 101b is interfaced with a storage unit 108 for storing the number of fetal movements with a corresponding timestamp. The fetal motion counting unit 101 is programmed with a fetal motion count algorithm in the fetal movement count trender 101b for comparing the measured number of fetal motions in a particular duration of time with the preset value of fetal motions. After comparison, the fetal motion counting unit 101 displays a message about fetus health based on the output of the comparison. In an embodiment of the present invention, the fetal movement counting unit 101 optionally comprises an input member actuatable by the user during each movement of the fetus for charting movement of the fetus.

In an embodiment of the present invention, a fetal motion counting unit 101 is made automatic using a plurality of motion sensors 101a which automatically measure the movements of the fetus and store them=with the corresponding timestamp. The automatic system works similar to a pedometer to sense the movements of the fetus. The handheld device 100 is tied to the belly/abdomen portion of the pregnant woman to sense approximate fetus movements. The accuracy of measurement is dependent on the optimization of the unit 101; the optimization is based on size of the pregnant woman's belly.

The ultrasound transducer unit 102 is provided in the handheld device 100 for recording the heartbeat of the fetus. The ultrasound transducer unit 102 comprises ultrasound crystals for generating the waves of ultrasound frequency. The ultrasound transducer unit 102 further comprises a transmitter interfaced with the ultrasound crystals for transmitting the waves of ultrasound frequency to the womb, and a receiver for receiving the signals from the womb for recording the heartbeat of the fetus. The receiver is interfaced with the storage unit for storing the heartbeat of the fetus. The receiver is also connected with the transducer and the audio amplifier for converting the ultrasound frequency into sound and amplifying the signals. The ultrasound transducer unit 102 further comprises an audio member operable as a speaker connected to the audio amplifier for playing the heartbeat sound.

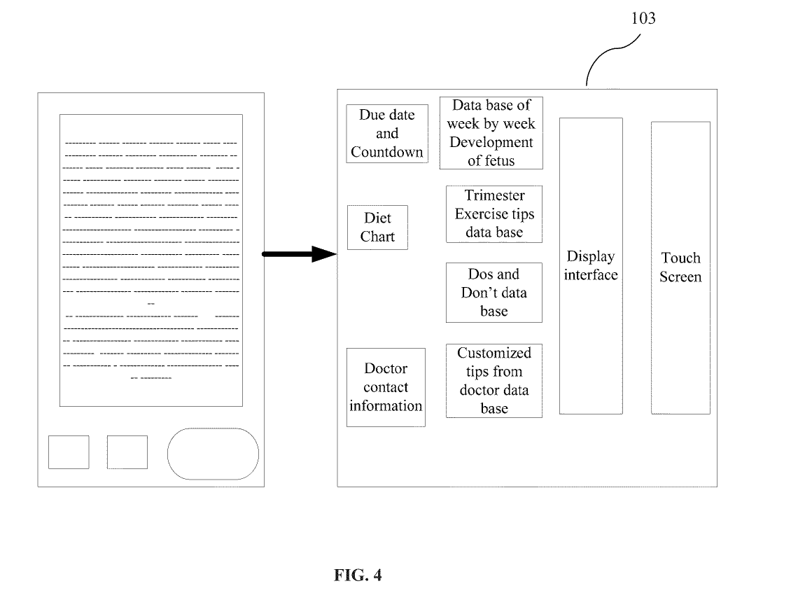

The display interface 103 with a knowledge database as shown in the FIG. 4 is provided in the handheld device 100. A display interface 103 is adopted for displaying the information stored in the knowledge database that is required to educate a user about health tips, and to help the user in controlling mood swings. The knowledge database comprises stored data of various parameters which helps users to educate themselves during pregnancy. The knowledge database replaces heavy books used conventionally. The various parameters which can be helpful to the user during pregnancy includes but is not limited to, due date and count downs of pregnancy, diet charts for the user, information about week-by-week development of the baby, the doctor's detailed contact information, trimester exercise tips and customized tips from the doctor. In the handheld device 100 according to embodiments of the present invention, the user is able to access all of the above information using a display interface 103 provided in the display. Hence, the incorporation of the display interface 103 with the knowledge database reduces the burden on the pregnant woman who must otherwise carry bulky books to educate herself on controlling mood swings, diet, and other aspects of pregnancy.

The user has the ability to press the input button present on the display interface 103, and the various options can be selected from a group comprising: due date count downs, doctor contact information, diet chart, trimester exercise tips, do's and don'ts information, and customized tips from doctor database. The user can select any of the above mentioned options to read the data related to the selected option.

The temperature sensor 104 is provided in the handheld device 100 for measuring the body temperature of the pregnant woman. Maintaining the body temperature of a pregnant woman within optimal limits is very important during pregnancy. If the temperature of a pregnant woman exceeds the prescribed temperature then it will affect fetal health, hence it is recommended that pregnant women maintain a body temperature within the optimal limits. The recommended body temperature for a pregnant woman is 102.2° F. Thus, the pregnant woman should not let her core body temperature rise above 102.2° F. Hence, a handheld device 100 is equipped with a temperature sensor 104 for measuring the temperature of the user and notifies the pregnant woman about her body temperature. This can be of great help to the pregnant for maintaining their body temperature within optimal ranges, which would in turn result in healthy fetal growth. In an embodiment of the present invention, the temperature sensor 104 is selected from at least one of an infrared radiation temperature sensor, thermocouples, and thermo meters.

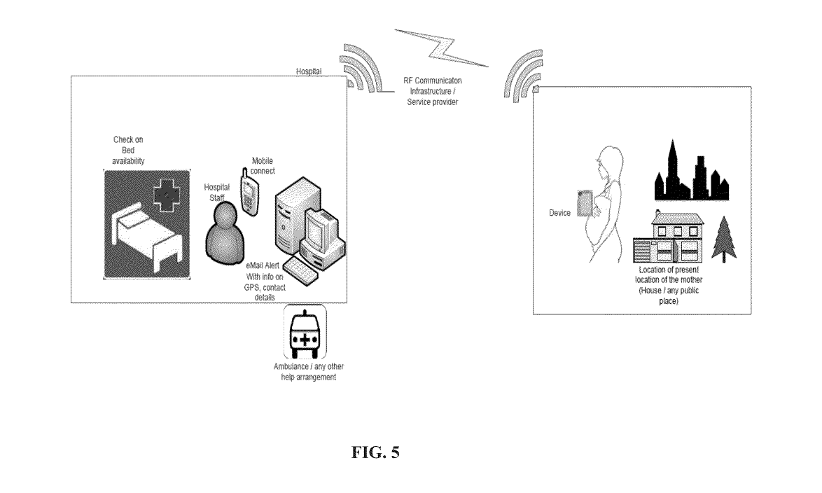

The alert system 105 is provided in the handheld device as shown in FIG. 5 for sending a preconfigured emergency message to a hospital management system or mobile device of a care giver, mid-wife, or partner of the user upon actuation of the alert system 105 by the user. The alert system 105 comprises a button actuable by the user during emergency, and upon actuation of the button, the alarm activation unit sends a preconfigured message through a Radio Frequency communication infrastructure provided in the handheld device 100. The communication infrastructure communicates with at least one of an Electronic Fetal Monitor of the hospital management system or mobile phone of the midwife or partner through a Global Positioning System (GPS) to send the preconfigured message. In one embodiment of the present invention, the emergency message can be in the form of, but not limited to, SMS, voice SMS, call or e-mail.

The audio recorder 106 is provided in the handheld device for recording the health conditions of the user. The audio recorder 106 comprises an input button actuatable by the user; upon actuation of the input button, the audio recorder records the health conditions of the user. The audio recorder 106 is interfaced with the storage unit 108 for storing the recorded data.

In an embodiment of the present invention, the handheld device is further comprises a user interface having at least one of soft buttons and hard keys for recording health conditions of the user. The user interface provided for recording the health condition of the user would be of great help to a pregnant woman.

The handheld device 100 is further equipped with a data communication media interface 107 for downloading data stored in the storage unit 108 to the Electronic Fetal Health monitor of the hospital management system or any other external interface. The communication media interface 107 can be selected from at least one of a wired and wireless communication media. In an embodiment of the present invention, wired communication media is selected from group comprising, but not limited to, a data port, a USB port, a HDMI port, and a LAN port. In another embodiment of the present invention, wireless transmission media is selected from at least one of blue tooth, Infrared, and WI-FI connections.

A care giver can download the data stored in the storage unit 108, using the communication media interface 107, to the Electronic fetal Monitor when the user visits the care giver with the handheld device 100. A care giver can connect the handheld device 100 to the Electronic Fetal Monitor and, upon activating the transfer button, the data stored in the storage unit is transferred to the Electronic Fetal Monitor. In an embodiment of the present invention, the data stored in the storage unit 108 includes, but is not limited to, fetal movements, heartbeat sounds and the health conditions of the user carrying the fetus.

The handheld device 100 is compatible with an Electronic Fetal Monitor associated with a service provider. The handheld device 100 is given to the pregnant woman by the service provider when the pregnant woman comes to the hospital for a pregnancy check-up. The handheld device 100 remains with the user at home during the pregnancy. After the delivery, the option is provided to the pregnant woman to return the device to the service provider.

In an embodiment of the present invention, the handheld device 100 can be powered using at least one of a battery, a solar power source and any other source which serves the purpose.

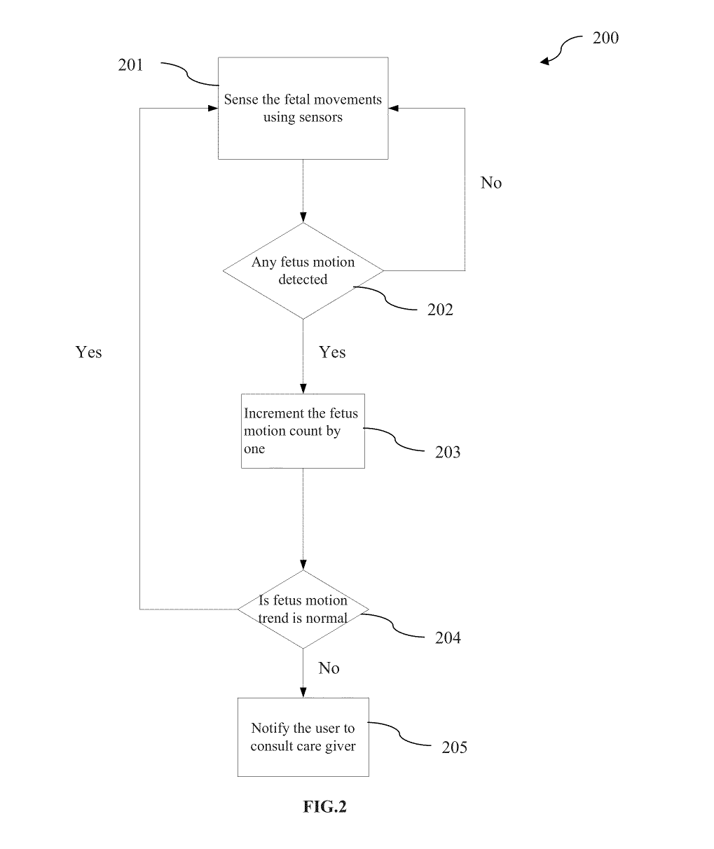

FIG. 2 is an exemplary embodiment of the present invention which illustrates a flow chart showing an operational sequence of a fetal motion counting unit 101 of the handheld device 100. The fetal motion counting unit 101 checks for fetal motion using at least one motion sensor 101a as shown in step 201. If fetal motion is detected, then the device 100 increases the fetal movement count by one and stores the count in the storage unit 108 with a timestamp as shown in step 202. If fetal motion is not detected, then the unit performs the step 201. Then, the unit 101 compares the measured values of fetal movement along with the timestamp with the preset values of fetal movement as shown in step 203. If the fetal motion detected by the device is normal then the unit performs step 201. If the fetal motion detected by the device is not normal, the unit 101 notifies the user to consult the care giver for fetal health monitoring as shown in step 204.

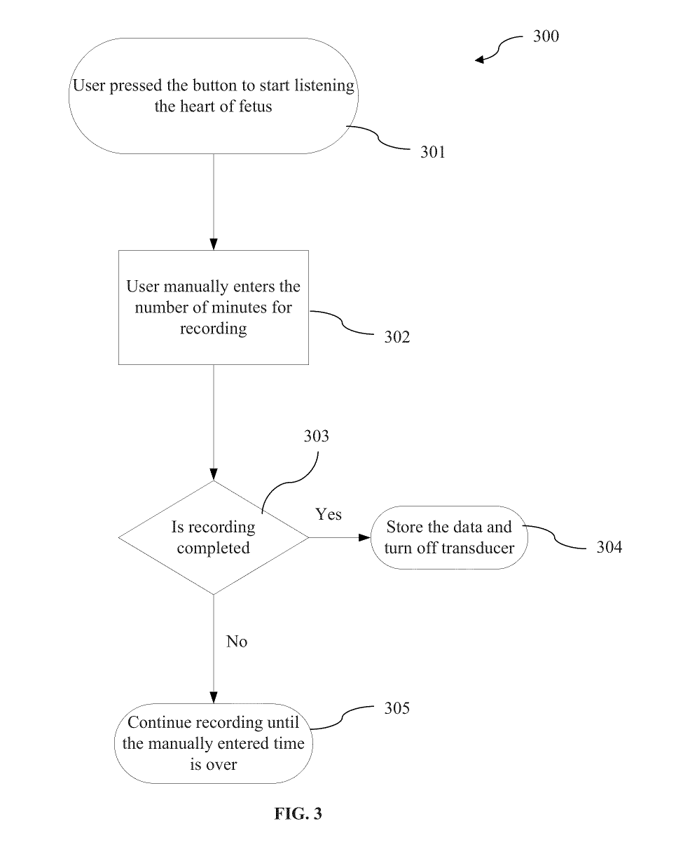

FIG. 3 is an exemplary embodiment of the present invention illustrating a flow chart showing an operational sequence of an ultrasound transducer unit 102 of the handheld device 100. The user will activate the ultrasound transducer 102 for recording the heartbeat of the fetus as shown in step 301, and the user will then set the time for controlling the ultrasound transducer 102 using the timer as shown in step 302. The ultrasound transducer 102 stores the heartbeat sound in the storage unit 108 once recording is completed as shown in step 303, or the unit ultrasound transducer 102 will record the heart beat until the set time expires as shown in step 304.

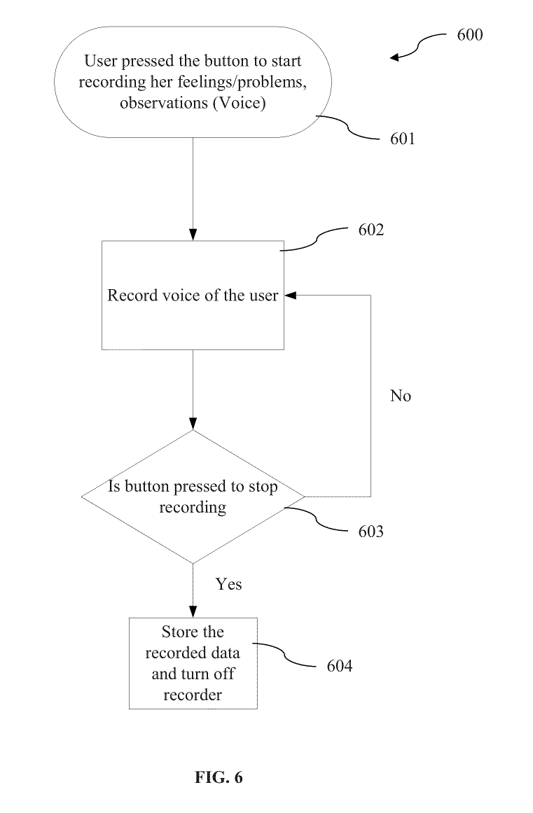

FIG. 6 is an exemplary embodiment of the present invention illustrating a flow chart showing an operational sequence of an audio recorder 106 of the handheld device 100. The user activates the audio recorder 106 using a button for recording the health conditions of the user carrying the fetus as shown in step 601. The audio recorder 106 then records the health conditions of the user as shown in step 602. Then, the control unit frequently checks for the activation of a stop button to stop the audio recorder 106 as shown in step 603. If the stop button is activated, the control unit stores the recorded sound in the storage unit 108 as shown in step 604. If the stop button is not activated, the system continues recording as shown in step 602.

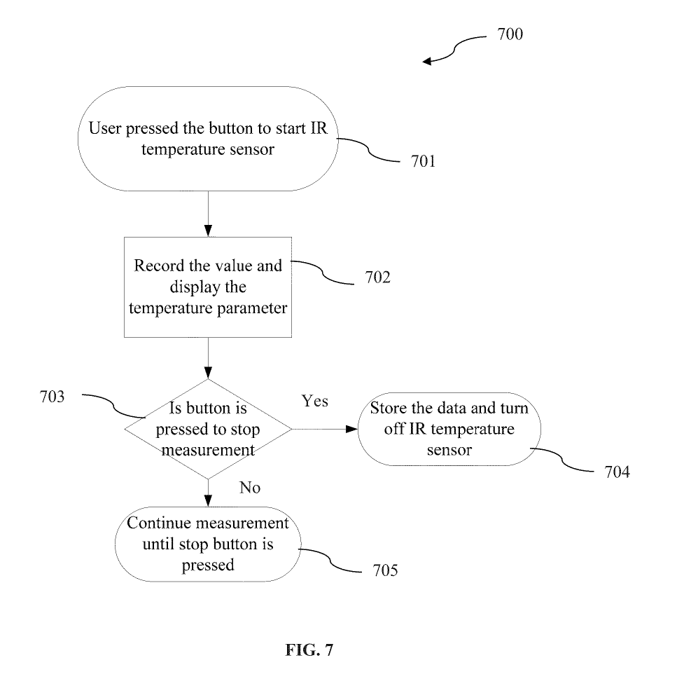

FIG. 7 is an exemplary embodiment of the present invention illustrating a flow chart showing an operational sequence of a temperature sensor 104 of the handheld device 100. The user activates the temperature sensor 104 using a button for measuring the temperature of the user as shown in step 701. Then, the control unit measures the temperature and displays the measured value of temperature as shown in step 702. Then, the control unit frequently checks for activation of a stop button to stop the temperature as shown in step 703. If the stop button is activated the control unit stores the recorded temperature in the storage unit 108 as shown in step 704. If the stop button is not activated, the system continues recording as shown in step 705.

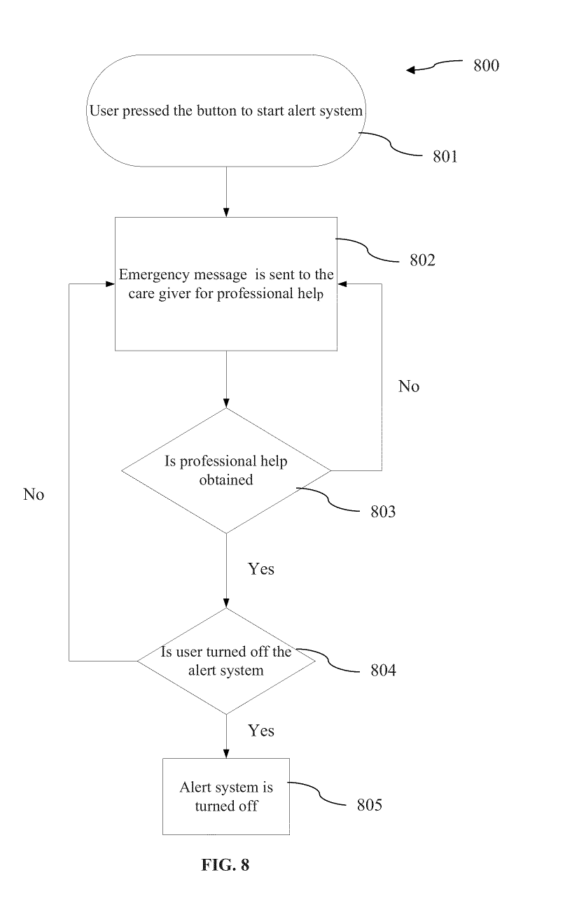

FIG. 8 is an exemplary embodiment of the present invention illustrating a flow chart showing an operational sequence of an alert system 105 of the handheld device 100. The user activates the alert system 105 using a button as shown in step 801. The unit then sends a preconfigured emergency message to a hospital management system or mobile device of a mid-wife, care giver, or partner as shown in step 802. Then the control unit checks for completion of professional assistance as shown in step 803. If the professional assistance is provided then the user turns off the button as shown in 804. If professional assistance is not provided, the unit performs step 802. Then, the alert system 105 is turned off and main menu is returned as shown in step 805.

Thus, embodiments of the present invention provide a handheld device which acts as a personal health device for pregnant women. The handheld device helps the pregnant woman analyze fetal growth and well-being from home. Further, the handheld device educates the mother about her diet and helps to control her mood swings.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A handheld device for fetal health monitoring, the device comprising:
at least one sensor configured to detect fetal movement;
an ultrasound transducer unit configured to record a heartbeat sound of the fetus; and
a storage unit connectable to the at least one sensor and to the ultrasound transducer unit, and configured to store the detected fetal movement along with a timestamp and the heartbeat sound of the fetus in the form of digital information.

2. The handheld device of claim 1, wherein the ultrasound transducer unit is configured to interface with a timer set by a user to control the ultrasound transducer.

3. The handheld device of claim 1, wherein the ultrasound transducer unit further comprises an audio member operable as a speaker to generate the heartbeat sound.

4. The handheld device of claim 1, further comprising a temperature sensor configured to measure the temperature of a pregnant woman.

5. The handheld device of claim 1, further comprising an alert system actuatable by a user, the alert system being configured to send an emergency message to at least one of a hospital management system and a mobile device of a care giver.

6. The handheld device of claim 1, further comprising at least one of an audio recorder and a user interface having at least one of soft buttons and hard keys for recording health conditions of a user, wherein the audio recorder and user interface are interfaced with the storage unit.

7. The handheld device of claim 1, further comprising a display interface for displaying information related to at least one of pregnancy, diet tips, a doctor's contact information, due date and count down, and customized tips from a doctor stored in a knowledge database for educating the user.

8. The handheld device of claim 1, further comprising a communication media interface configured to transmit data stored in the storage unit to an Electronic Fetal Monitor associated with a service provider.

9. A method of monitoring fetal health, the method comprising:
detecting fetal movement using at least one sensor;
recording a heartbeat sound of the fetus using an ultrasound transducer unit; and
storing the detected fetal movement along with a timestamp and the heartbeat sound of the fetus in the form of digital information in a storage unit.

10. The method of claim 9, further comprising comparing the stored fetal movements with a preset value of fetal movements; and
notifying a user about fetal health based on the comparison.

11. The method of claim 9, further comprising transmitting, using a communication media interface, data stored in the storage unit to an Electronic Fetal Monitor associated with a service provider.

12. The method of claim 9, further comprising measuring the temperature of a pregnant woman using a temperature sensor.

13. The method of claim 9, further comprising displaying, using a display interface, information related to at least one of pregnancy, diet tips, a doctor's contact information, due date and count down, and customized tips from a doctor stored in a knowledge database.

14. The method of claim 9, further comprising sending an emergency message to at least one of a hospital management system and a mobile device of a care giver upon actuation of an alert system by a user.

15. The method of claim 9, further comprising:
recording the health conditions of a user using at least one of an audio recorder and a user interface having at least one of soft buttons and hard keys; and
storing the recorded health conditions in the storage unit.
